(19) United States
(12) Patent Application Publication
(10) Pub. No.: US 2024/0218565 A1
(43) Pub. Date: Jul. 4, 2024

(54) PLANT MATERIAL RECYCLING INOCULANT AND USES THEREOF

(71) Applicant: IMIO TECHNOLOGIES, INC., Essex Junction, VT (US)

(72) Inventors: Victoria I. HOLDEN, Essex Junction, VT (US); Charles Smith, Essex Junction, VT (US)

(73) Assignee: IMIO TECHNOLOGIES, INC., Essex Junction, VT (US)

(21) Appl. No.: 18/542,091

(22) Filed: Dec. 15, 2023

Related U.S. Application Data
(63) Continuation-in-part of application No. 18/259,683, filed on Jun. 28, 2023.
(60) Provisional application No. 63/229,172, filed on Aug. 4, 2021.
(60) Provisional application No. 63/132,185, filed on Dec. 30, 2020.

(51) Int. Cl. D01C 1/04; C12N 1/14; C12N 1/18; C12N 1/20; C12R 1/125; C12R 1/23; C12R 1/24; C12R 1/245; C12R 1/25; C12R 1/46; C12R 1/465; C12R 1/86; C12R 1/865; C12R 1/885

(52) U.S. Cl. CPC D01C 1/04; C12N 1/14; C12N 1/18; C12N 1/20; C12R 2001/125; C12R 2001/23; C12R 2001/24; C12R 2001/245; C12R 2001/25; C12R 2001/46; C12R 2001/465; C12R 2001/86; C12R 2001/865; C12R 2001/885

(57) ABSTRACT

Disclosed are microbial compositions having unique combinations of microbial species which are used in plant material recycling and retting. Also disclosed microbial inoculants which contains such a microbial composition, water, and an optional carbon source. The microbial compositions and microbial inoculants are particularly useful in recycling high lignin-content plant material. Methods of making and methods of using the microbial compositions and the microbial inoculants are also described.

### CROSS-REFERENCE TO RELATED APPLICATIONS

This application is a Continuation-in-Part (CIP) application of application Ser. No. 18/259,983, filed on Jun. 28, 2023; which is a national phase of PCT International Application No. PCT/US2021/057269, filed on Oct. 29, 2021; which claims priority to U.S. Provisional Application No. 63/132,185 filed on Dec. 30, 2020, and to U.S. Provisional Application No. 63/229,172 filed on Aug. 4, 2021, the disclosures of which are all incorporated by reference.

### STATEMENT OF U.S. GOVERNMENT SUPPORT

This invention was made with government support under grant award number (FAIN) 2014792 awarded by the Office of National Science Foundation (NSF). The U.S. government has certain rights in the invention.

### FIELD OF THE INVENTION

The invention relates to microbial compositions and microbial inoculants having unique combinations of microbial species which are used in plant material recycling to form a nutrient source. The microbial compositions and microbial inoculants are particularly useful in recycling high lignin-content plant material. The invention also relates to methods of making and methods of using the microbial compositions and the microbial inoculants.

### BACKGROUND

Each year the global agricultural industry consumes approximately $376B in fertilizer while generating over 12B tons of wasted plant material (1). Both practices contribute significantly to climate change, generating an estimated 924M tons of CO₂ equivalent greenhouse gas emissions annually (2). The agricultural sector is the 5^{th} leading contributor to US greenhouse gas emissions, generating 9% of emissions (3). While much of this can be traced to large-scale livestock operations, the UN estimates that mishandled crop residuals generated an estimated 31M metric tons of CO₂ equivalent greenhouse gas emissions in the US as farmers allow leftover plant material to rot in their fields or dispose of it in burn piles or landfills (2).

Synthetic fertilizer also causes a litany of environmental harms across each phase of its lifecycle, from its production that uses fracked natural gas, to its transportation to farms that results in greenhouse gas emission, and its runoff into local waterways that causes cyanobacterial blooms and negatively impacts drinking water quality. In addition to harming the environment, the large-scale use of synthetic fertilizers and common disposal methods of burning and landfilling crop residuals overlook the massive potential of leftover plant material to return nutrients to the fields as an effective alternative to these practices.

The global agriculture industry is a dynamic industry as farmers worldwide shift crops to meet demand and begin farming new commercial crops, such as Cannabis sativa, certain strains of which are known as hemp and others which contain THC or other cannabinoids. In the US, Cannabis farmers spent approximately $31.47M on fertilizer purchases in 2019. These projections were derived by multiplying the 288,000 acres of Cannabis planted in 2019 by $111.60, the modeled per acre nutrient cost (4,5). This per acre nutrient cost is a conservative estimate based on conversations with Cannabis farmers and other industry participants who posit that nutrient costs typically range from $300-$1,000 per acre. US Cannabis cultivation is projected to increase at a rapid pace in the years to come, making this a pivotal time in setting the crop's trajectory for environmental benefit or harm. Cannabis demand is increasing based on consumer and medicinal interest in cannabinoids, and numerous applications for hemp varietals in health food, textile, and personal care end-markets; manufacturers increasingly turn to Cannabis as an environmentally friendly nutrient source, cotton alternative, and fatty acid source for products in these markets. It is currently expected that fertilizer costs will grow roughly in line with acreage increases, and therefore that Cannabis cultivators will spend hundreds of millions of dollars on fertilizer in the next several years.

As Cannabis cultivation acreage increases, so too will the volume of wasted plant material and subsequent greenhouse gas emissions from improper disposal. In agriculture, “harvest index” measures the ratio of plant yield to total plant material for a given crop (6). In estimating the expected volume of waste per acre, a harvest index of 0.6 is applied to the average yield of 1,500 lbs. of sellable plant material per planted acre (5). This implies 1,000 lbs. of waste per acre, amounting to 288M lbs. of wasted plant material across the US in 2019, and billions of pounds of annual waste by 2025 based on market growth forecasts. A harvest index of 0.6 is consistent with the experimental data generated by growers using diligent cultivation practices and periodic pruning to produce a greater density of sellable plant material and a higher harvest index. A harvest index of 0.6 is likely much higher than the industry average, therefore building substantial conservatism into our waste generation estimates. One study found that average harvest indices for industrial Cannabis range from 0.06-0.23, indicating a total volume of wasted plant material between 1.44-6.77B lbs. for 2019.

Like other woody plants, the major structural compounds that make up a Cannabis plant include lignin, cellulose, and hemicellulose (7). Lignin is a complex, heterogeneous polymer that is hydrophobic and rich in aromatic subunits that are difficult to break down (8). Cellulose, (C₆H₁₀O₅)ₙ, is a linear chain of hundreds to thousands of D-glucose units that is crystalline, strong, and resistant to hydrolysis (9). Hemicellulose is a heteropolymer that differs from cellulose because it is shorter and formed from different monosaccharides, such as xylose or mannose (10,11). Cannabis contains both bast (inner bark) and hurd (woody core) fibers that each have their own composition of lignin, cellulose, and hemicellulose. Bast fibers of Cannabis make up 20-40% of the plant and are composed of 5-9% lignin, 57-77% cellulose, and 9-14% hemicellulose (12). Hurd fibers account for the other 60-80% of the plant and are composed of 21-24% lignin, 40-48% cellulose, and 18-24% hemicellulose (12). Lignin, cellulose, and hemicellulose are enzymatically degraded by peroxidases, cellulases, and hemicellulases, respectively (8-10).

The Cannabis stalks pose a significant technical challenge to organic recycling processes due to their woody nature and high concentration of lignin. These stalks are a reservoir of valuable nutrients as they store approximately 80% of the nitrogen that Cannabis plants consume (13). Whereas the durability of lignin benefits living plants, it inhibits recycling and nutrient acquisition by most microbes (8,14).

Microorganisms are the engine of organics recycling and are key determinants of soil and plant health (15,16). Traditional composting methods take a roundabout approach to cultivating their desired microbial communities, relying on a continuously managed balance of carbon and nitrogen feedstocks and the maintenance of specific temperature and moisture levels through regular turning and watering (15). An effective thermophilic compost pile requires a succession of multiple microbial communities to decompose organic matter over the course of many months (15,17,18). Even for farmers willing to invest time, labor, and resources to pursue the correct input balance, the consistency of this microbial succession and the output of the process is variable (15).

There is a need for a more direct approach to plant material recycling—one that requires less manual labor and reduces recycling time. More particularly the need exists for recycling aids and processes which break down what would be otherwise wasted Cannabis and other high-lignin-content plant material into bioavailable nutrients to nourish new plants. This invention answers such needs.

### SUMMARY OF THE INVENTION

This invention relates to microbial compositions having unique combinations of microbial species which are used in plant material recycling to form a nutrient source. A microbial composition of the invention is particularly useful in recycling high lignin plant material.

One microbial composition of the invention is a mixture of microbial species comprising, consisting essentially of, or consisting of:
at least one first microbial species selected from the group consisting of Lactobacillus acidophilus, Saccharomyces pastorianus, Lactobacillus brevis, Streptococcus thermophilus, Butyrivibrio fibrisolvens, Pseudomonas putida, Rhodococcus jostii, Trichoderma reesei, Phanerochaete chrysosporium, Irpex lacteus, Bacillus subtilis, Amycolatopsis spp., Acinetobacter spp., Cellulomonas fimi, Cellulomonas flavigena, Sphingomonas paucimobilis, Streptomyces coelicolor, and Irpex flavus; and at least one second microbial species selected from the group consisting of Lactobacillus casei, Lactobacillus plantarum, Lactobacillus fermentum, Lactobacillus delbrueckii, Bacillus subtilis, Saccharomyces cerevisiae, Rhodopseudomonas palustris, and Acetobacter spp.

Preferred microbial compositions according to this organization are those where a first microbial species contains at least one or more of P. putida, P. chrysosporium, I. lacteus, and S. coelicolor in combination with a second microbial species containing at least one of R. palustris and one or more of the identified Lactobacillus spp. A preferred microbial composition according to this organization is one where the at least one first microbial species are P. putida, P. chrysosporium, I. lacteus, and S. coelicolor, and the at least one second microbial species are R. palustris and at least one Lactobacillus spp.

Another microbial composition is a mixture of microbial species comprising:
at least one first microbial species selected from the group consisting of Lactobacillus casei, Lactobacillus plantarum, Lactobacillus fermentum, Lactobacillus delbrueckii, Bacillus subtilis, Rhodopseudomonas palustris, Acetobacter spp., Lactobacillus acidophilus, Saccharomyces pastorianus, Lactobacillus brevis, Streptococcus thermophilus, Butyrivibrio fibrisolvens, Pseudomonas putida, Rhodococcus jostii, Bacillus subtilis, Amycolatopsis spp., Acinetobacter spp., Cellulomonas fimi, Cellulomonas flavigena, Sphingomonas paucimobilis, Streptomyces coelicolor; and at least one second microbial species selected from the group consisting of Saccharomyces cerevisiae, Trichoderma reesei, Phanerochaete chrysosporium, Irpex lacteus, and Irpex flavus.
   Preferred microbial compositions according to this organization are those where a first microbial species contains at least one or more of R. palustris, S. coelicolor, and one or more of the identified Lactobacillus spp. in combination with a second microbial species containing at least one of P. chrysosporium, I. lacteus, and I. flavus. A preferred microbial composition according to this organization is one where the at least one first microbial species are R. palustris, S. coelicolor, and at least one Lactobacillus spp., and the at least one second microbial species are P. chrysosporium, I. lacteus, and I. flavus.

The invention also relates to a microbial inoculant which contains a microbial composition having a mixture of microbial species according to the invention, water and an optional carbon source.

The invention provides a method of preparing a microbial inoculant comprising the steps of resuspending a lyophilized mixture of microbial species according to the invention in water, and optionally adding a carbon source.

The invention provides a method for recycling plant material comprising the steps of contacting plant material to be recycled with a microbial composition of the invention or with a microbial inoculant of the invention to form an inoculated plant material, and enclosing (e.g., covering, sealing, placing in a container) the inoculated plant material for at least about two weeks to form a recycled plant material. The method may also include the step of applying the recycled plant material to a plant or a field.

### BRIEF DESCRIPTION OF THE DRAWINGS

FIG. 1 depicts results of biomass recycled with different microbial inoculants of the invention (FCM1-4) analyzed for peroxidase activity. Neg ctrl=negative control; EM=state-of-the-art industry standard.

FIG. 2 depicts results of biomass recycled with different microbial inoculants of the invention (FCM1-4) analyzed for phenol oxidase (phenoxidase) activity. Neg ctrl=negative control; EM=state-of-the-art industry standard.

FIG. 3 depicts results of biomass recycled with different microbial inoculants of the invention (FCM1-4) analyzed for beta-glucosidase (BG) activity. Neg ctrl=negative control; EM=state-of-the-art industry standard.

FIG. 4 depicts results of biomass recycled with different microbial inoculants of the invention (FCM1-4) analyzed for phosphatase (PHOS) activity. Neg ctrl=negative control; EM=state-of-the-art industry standard.

FIG. 5 depicts results of biomass recycled with different microbial inoculants of the invention (FCM1-4) analyzed for leucyl-aminopeptidase (LAP) activity. Neg ctrl=negative control; EM=state-of-the-art industry standard.

FIG. 6 depicts results of biomass recycled with different microbial inoculants of the invention (FCM1-4) analyzed for N-acetylglutamate synthase (NAG) activity. Neg ctrl=negative control; EM=state-of-the-art industry standard.

FIG. 7 shows the results of fresh biomass recycled with different microbial inoculants of the invention (FCM1-4) analyzed for pathogen suppression activity via plate competition assay. EM=state-of-the-art industry standard.

FIG. 8 shows the results of cured biomass recycled with different microbial inoculants of the invention (FCM1-4) analyzed for pathogen suppression activity via plate competition assay. EM=state-of-the-art industry standard.

FIGS. 9A-9B depicts results of cress grown in fresh (9A) and cured (9B) biomass recycled with different microbial inoculants of the invention (FCM1-4) analyzed for germination rate. EM=state-of-the-art industry standard.

FIGS. 10A-10B illustrate growth (10A) and leaf surface area (10B) for cress grown in biomass recycled with a microbial inoculant of the invention and cured in coco-coir (Coir+FCM Recycled Cannabis).

FIGS. 11A-11F depict results of soil cured with hemp biomass recycled with a microbial inoculant of the invention (FCM+Base soil) analyzed for pH (11A), organic matter content (11B), potassium content (11C), calcium and magnesium content (11D), phosphorous content (11E), and micronutrient content (11F).

FIGS. 12A-12F depict results of soil cured with hemp biomass recycled with a microbial inoculant-bran (FCM Bran+Base soil) analyzed for pH (12A), organic matter content (12B), potassium content (12C), calcium and magnesium content (12D), phosphorous content (12E), and micronutrient content (12F). EM=state-of-the-art industry standard.

FIG. 13 depicts results of rye straw recycled with a microbial inoculant of the invention analyzed for lignin content.

FIGS. 14A-14D depict results of transplanted hemp grown in unused hemp biomass recycled with a microbial inoculant of the invention analyzed for whole plant fresh weight (14A), bud dry weight (14B), bed/fresh weight ratio (14C), and % yield versus a control sample (14D). HH=Sour Hawaiian Haze hemp; SS=Sour Special Sauce hemp.

FIGS. 15A-15C depict results of a variety of non-hemp lignin-rich inputs recycled with a microbial inoculant of the invention and analyzed for ammonium nitrogen content (NH4-N; 15A), nitrate nitrogen content (NO3-N; 15B), and carbon to nitrogen ratio (C to N Ratio; 15C).

FIGS. 16A-16B depict results of a variety of non-hemp lignin-rich inputs recycled with a microbial inoculant of the invention and analyzed for phosphorous content (16A) and potassium (16B) content.

### DETAILED DESCRIPTION

This invention relates to microbial compositions having unique combinations of microbial species which are used in plant material recycling to form a natural nutrient source. This natural nutrient source can be used as a natural fertilizer, fertilizer substitute, fertilizer amendment, or fertilizer supplement. A microbial composition of the invention may comprise, consist essentially of, or consist of a mixture of microbial species as described here. A microbial composition of the invention may have mixtures of microbial species that are particularly suited for high lignin plant materials (for example, Cannabis plant material). Examples of other high lignin plant material include but are not limited to hops, ornamental flowers (roses, orchids, lavender, lilies, geranium, marigold), saffron, nursery and/or landscaping clippings from trees and bushes, Christmas trees (fir trees), wine grapes, sunflowers, broccoli, rice, tomatoes, sugar cane, corn, wheat, soy, cotton, home garden residuals, deciduous leaves, palm fronds, and tea (C. sinensis var. sinensis and C. s. var. assamica). In a microbial composition of microbial species, several strains of a given species can be present. A microbial composition of microbial species may also be a lyophilized microbial composition of the microbial species. The invention also relates to a microbial inoculant which contains a microbial composition having mixture of microbial species according to the invention, water, and an optional carbon source.

Introducing a microbial inoculant containing a mixture of microbial species according to the invention to leftover plant material to be recycled accelerates organics recycling and increases output consistency compared to traditional composting. Advantages of employing a recycling inoculant include, for example, a reduction in time and labor required to recycle plant material into a usable nutrient source, and increasing the efficacy of the output as a nutrient source which produces healthier plants and greater crop yields. The deployment of a microbial inoculant of the invention optimized for Cannabis and other lignin-containing commercial crop recycling allows for the recycling and reuse of plant material that would otherwise be burned, left to rot, removed for disposal or potentially recycled through less environmentally and/or economically advantageous methods. Lignin provides structure to Cannabis plants and is resistant to recycling, therefore, its breakdown presents the key technical challenge in effectively recycling Cannabis waste. To address such a challenge, this invention is directed to microbial compositions and microbial inoculants having mixtures of microbes capable of producing enzymes that specifically degrade lignin and the other compounds that give plants such as Cannabis their structure and rigidity. Furthermore, the invention relates to growing, lyophilizing, and combining mixtures of microbes to form a microbial composition that can be reconstituted into an aqueous suspension that contacts (is mixed with or applied directly to) leftover plant material to be recycled. When enclosed to form an environment to facilitate the microbial activity (such as a microaerobic or an anaerobic environment), the recycling of plant material by the microbes results in a nutrient rich product that can be repurposed as a natural nutrient source.

One microbial composition of the invention is a mixture of microbial species comprising:
at least one first microbial species selected from the group consisting of Lactobacillus acidophilus, Saccharomyces pastorianus, Lactobacillus brevis, Streptococcus thermophilus, Butyrivibrio fibrisolvens, Pseudomonas putida, Rhodococcus jostii, Trichoderma reesei, Phanerochaete chrysosporium, Irpex lacteus, Bacillus subtilis, Amycolatopsis spp., Acinetobacter spp., Cellulomonas fimi, Cellulomonas flavigena, Sphingomonas paucimobilis, Streptomyces coelicolor, and Irpex flavus; and at least one second microbial species selected from the group consisting of Lactobacillus casei, Lactobacillus plantarum, Lactobacillus fermentum, Lactobacillus delbrueckii, Bacillus subtilis, Saccharomyces cerevisiae, Rhodopseudomonas palustris, and Acetobacter spp.

Preferred microbial compositions according to this organization are those where a first microbial species contains at least one or more of P. putida, P. chrysosporium, I. lacteus, and S. coelicolor in combination with a second microbial species containing at least one of R. palustris and one or more of the identified Lactobacillus spp. A preferred microbial composition according to this organization is one where the at least one first microbial species are P. putida, P. chrysosporium, I. lacteus, and S. coelicolor, and the at least one second microbial species are R. palustris and at least one Lactobacillus spp.

Another microbial composition is a mixture of microbial species comprising:
at least one first microbial species selected from the group consisting of Lactobacillus casei, Lactobacillus plantarum, Lactobacillus fermentum, Lactobacillus delbrueckii, Bacillus subtilis, Rhodopseudomonas palustris, Acetobacter spp., Lactobacillus acidophilus, Saccharomyces pastorianus, Lactobacillus brevis, Streptococcus thermophilus, Butyrivibrio fibrisolvens, Pseudomonas putida, Rhodococcus jostii, Bacillus subtilis, Amycolatopsis spp., Acinetobacter spp., Cellulomonas fimi, Cellulomonas flavigena, Sphingomonas paucimobilis, Streptomyces coelicolor; and
at least one second microbial species selected from the group consisting of Saccharomyces cerevisiae, Trichoderma reesei, Phanerochaete chrysosporium, Irpex lacteus, and Irpex flavus.

Preferred microbial compositions according to this organization are those where a first microbial species contains at least one or more of R. palustris, S. coelicolor, and one or more of the identified Lactobacillus spp. in combination with a second microbial species containing at least one of P. chrysosporium, I. lacteus, and I. flavus. A preferred microbial composition according to this organization are those where a first microbial species contains at least one or more of R. palustris, S. coelicolor, and one or more of the identified Lactobacillus spp. in combination with a second microbial species containing at least one of P. chrysosporium, I. lacteus, and I. flavus.

A preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, and Phanerochaete chrysosporium.

Another preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, and Irpex lacteus.

Another preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, and Irpex lacteus.

Another preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, Irpex lacteus, and Pseudomonas putida.

Another preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, and Pseudomonas putida.

Another preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, and Pseudomonas putida.

Another preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, Irpex lacteus, and Pseudomonas putida.

Another preferred microbial composition of the invention is Lactobacillus casei, Lactobacillus plantarum, Lactobacillus fermentum, Lactobacillus delbrueckii, Bacillus subtilis, Saccharomyces cerevisiae, and Rhodopseudomonas palustris.

Another preferred microbial composition of the invention is Acetobacter spp., Lactobacillus acidophilus, Saccharomyces pastorianus, Lactobacillus brevis, Streptococcus thermophilus, Butyrivibrio fibrisolvens, Rhodococcus jostii, an Amycolatopsis sp., an Acinetobacter sp., Cellulomonas fimi, Cellulomonas flavigena, Sphingomonas paucimobilis, Streptomyces coelicolor, Trichoderma reesei, and Irpex flavus.

Another preferred microbial composition of the invention is Lactobacillus acidophilus, Saccharomyces pastorianus, Lactobacillus brevis, Streptococcus thermophilus, Butyrivibrio fibrisolvens, Rhodococcus jostii, Trichoderma reesei, an Amycolatopsis sp., an Acinetobacter sp., Cellulomonas fimi, Cellulomonas flavigena, Sphingomonas paucimobilis, Streptomyces coelicolor, Irpex flavus and an Acetobacter sp.

A microbial composition or a microbial inoculant of the invention may contain microbial species capable of degrading lignin through the production of lignin-modifying enzymes that are predominantly peroxidases (8,19). Most microbial species that produce peroxidases are fungi; however, recent studies have identified multiple species of bacteria that encode and/or produce them (20).

The microbial species used in a microbial composition or a microbial inoculant of the inventions themselves can originate from a frozen glycerol stock, a solid-medium growth plate, or a commercially available source. A microbial species can also be isolated from environmental samples or purchased from open-access culture collections. The selected microbial species can then be streak-plated in a sterile environment on a petri dish or other containers of solid nutrient media to generate single colony isolates. Streak-plated samples on petri dishes or other containers can be incubated and isolated using techniques known in the art. For example, a microbial species may be incubated for 24-48 hours or longer at 30° C. aerobically, at 37° C. under 5% CO₂, or at any other condition optimal or sufficient for colony formation for a given species or strain. After incubation and colony formation, individual colonies can be isolated for propagation in liquid nutrient media for a further 24-48 hours or longer as stated above. Isolated microbial species or strains can be stored at −80° C. in 25-50% glycerol for continued propagation. Nutrient media and growth conditions can vary and are preferably optimized for a given strain. Nutrient media can be used for culturing, isolating, and storing microbes. Suitable nutrient media can be comprised of a carbon source, an amino acid source, salts, buffers, and yeast or meat extracts. Nutrient media can be prepared as a liquid or as a solid by supplementing with agar.

In a microbial composition or a microbial inoculant of the invention, individual strains or species can be present in equal concentrations. Alternatively, individual strains or species can be present in >1- to 1,000-fold excess over another strain or species present, in >1- to 500-fold excess, in >1- to 100-fold excess, in >1- to 50-fold excess or in >1- to 10-fold excess.

As discussed above a microbial composition containing a mixture of microbial species according to the invention may be a mixture of individually lyophilized microbial species. As known in the art lyophilization is a process by which water is removed by freezing the material and then reducing the pressure and adding heat to allow the frozen water in the material to sublimate. Lyophilization can be used to preserve perishable material, including microbes, and make it more convenient for transport. Preparation of the lyophilized mixture can be accomplished by inoculating, growing, pelleting, and lyophilizing individual species or strains before combining the lyophilized materials to form the lyophilized mixture. Strains of the same species can be combined after pelleting and before lyophilization, or after pelleting and lyophilization.

Starter cultures for lyophilization mixture can be prepared by inoculating a strain from a frozen glycerol stock or solid growth plate into liquid media for example using 5-500 mL volume or other volumes known in the art. Likewise, starter culture volume can be for example <5 mL or >500 mL or other volumes known in the art. Starter cultures can be used to inoculate a bulk culture that is for example 20-50 L in volume or other volumes known in the art. Likewise bulk culture can be for example <20 L or >50 L or other volumes known in the art. Bulk culture can be cycled through multiple draw/fill cycles as desired. Draw/fill cycles involve growing the culture to the desired cell density, removing a portion of the culture, and supplementing the remainder with fresh media for continued growth. Once desired cell density is reached, microbes can be pelleted from media by centrifugation. Strains of the same species can be optionally combined, and pellets can be resuspended in, for example, 2 L of media and lyophilized. Resuspension volume can be resuspended in for example volumes <2 L or >2 L or volumes depending on the capacity of lyophilization equipment. Individual lyophilized microbial species can be combined to generate the final lyophilized mixture. Lyophilized mixture can be packaged in packets for subsequent distribution and use.

This invention relates to a preparation of a microbial inoculant for recycling plant material, including high lignin content plant material. A microbial inoculant comprises, consists essentially of, or consists of a mixture of microbial species according to the invention, water, an optional carbon source. A recycling inoculant can be generated by resuspending a microbial composition in water. Resuspension volume can be for example <1 gallon or >1 gallon or other such volumes. This resuspension can optionally be supplemented with a carbon source, such as glucose or other sugars. The invention then also provides a method of preparing a microbial inoculant comprising the steps of resuspending a lyophilized mixture of microbial species according to the invention in water, and optionally adding a carbon source.

One microbial inoculant of the invention comprises:
at least one first microbial species selected from the group consisting of Lactobacillus acidophilus, Saccharomyces pastorianus, Lactobacillus brevis, Streptococcus thermophilus, Butyrivibrio fibrisolvens, Pseudomonas putida, Rhodococcus jostii, Trichoderma reesei, Phanerochaete chrysosporium, Irpex lacteus, Bacillus subtilis, Amycolatopsis spp., Acinetobacter spp., Cellulomonas fimi, Cellulomonas flavigena, Sphingomonas paucimobilis, Streptomyces coelicolor, and Irpex flavus;
at least one second microbial species selected from the group consisting of Lactobacillus casei, Lactobacillus plantarum, Lactobacillus fermentum, Lactobacillus delbrueckii, Bacillus subtilis, Saccharomyces cerevisiae, Rhodopseudomonas palustris, and Acetobacter spp.;
water; and
an optional carbon source.
   Preferred microbial inoculants according to this organization are those where a first microbial species contains at least one or more of P. putida, P. chrysosporium, I. lacteus, and S. coelicolor in combination with a second microbial species containing at least one of R. palustris and one or more of the identified Lactobacillus spp. A preferred microbial inoculant according to this organization is one where the at least one first microbial species are P. putida, P. chrysosporium, I. lacteus, and S. coelicolor, and the at least one second microbial species are R. palustris and at least one Lactobacillus spp.

Another microbial inoculant of the invention comprises
at least one first microbial species selected from the group consisting of Lactobacillus casei, Lactobacillus plantarum, Lactobacillus fermentum, Lactobacillus delbrueckii, Bacillus subtilis, Rhodopseudomonas palustris, Acetobacter spp., Lactobacillus acidophilus, Saccharomyces pastorianus, Lactobacillus brevis, Streptococcus thermophilus, Butyrivibrio fibrisolvens, Pseudomonas putida, Rhodococcus jostii, Bacillus subtilis, Amycolatopsis spp., Acinetobacter spp., Cellulomonas fimi, Cellulomonas flavigena, Sphingomonas paucimobilis, Streptomyces coelicolor;
at least one second microbial species selected from the group consisting of Saccharomyces cerevisiae, Trichoderma reesei, Phanerochaete chrysosporium, Irpex lacteus, and Irpex flavus;
water; and
an optional carbon source.

Preferred microbial inoculants according to this organization are those where a first microbial species contains at least one or more of R. palustris, S. coelicolor, and one or more of the identified Lactobacillus spp. in combination with a second microbial species containing at least one of P. chrysosporium, I. lacteus, and I. flavus. A preferred microbial inoculant according to this organization is one where the at least one first microbial species are R. palustris, S. coelicolor, and at least one Lactobacillus spp., and the at least one second microbial species are P. chrysosporium, I. lacteus, and I. flavus.

A preferred microbial inoculant of the invention are those wherein the microbial species are Lactobacillus casei, Lactobacillus plantarum, Lactobacillus fermentum, Lactobacillus delbrueckii, Bacillus subtilis, Saccharomyces cerevisiae, Rhodopseudomonas palustris, and Phanerochaete chrysosporium.

Another preferred microbial composition of the invention is Lactobacillus casei, Lactobacillus plantarum, Lactobacillus fermentum, Lactobacillus delbrueckii, Bacillus subtilis, Saccharomyces cerevisiae, Rhodopseudomonas palustris, and Irpex lacteus.

Another preferred microbial inoculant of the invention are those wherein the microbial species are Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, and Irpex lacteus.

Another preferred microbial inoculant of the invention are those wherein the microbial species are Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, Irpex lacteus, and Pseudomonas putida.

Another preferred microbial inoculant of the invention are those wherein the microbial species are Lactobacillus casei, Lactobacillus plantarum, Lactobacillus fermentum, Lactobacillus delbrueckii, Bacillus subtilis, Saccharomyces cerevisiae, Rhodopseudomonas palustris, and Pseudomonas putida.

Another preferred microbial inoculant of the invention are those wherein the microbial species are Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, and Pseudomonas putida.

Another preferred microbial inoculant of the invention are those wherein the microbial species are Lactobacillus casei, Lactobacillus plantarum, Lactobacillus fermentum, Lactobacillus delbrueckii, Bacillus subtilis, Saccharomyces cerevisiae, Rhodopseudomonas palustris, Irpex lacteus, and Pseudomonas putida.

Another preferred microbial inoculant of the invention are those wherein the microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris.

Another preferred microbial inoculant of the invention are those wherein the microbial species are Acetobacter spp., Lactobacillus acidophilus, Saccharomyces pastorianus, Lactobacillus brevis, Streptococcus thermophilus, Butyrivibrio fibrisolvens, Rhodococcus jostii, an Amycolatopsis sp., an Acinetobacter sp., Cellulomonas fimi, Cellulomonas flavigena, Sphingomonas paucimobilis, Streptomyces coelicolor, Trichoderma reesei, and Irpex flavus.

Another preferred microbial inoculant of the invention are those wherein the microbial species are Lactobacillus acidophilus, Saccharomyces pastorianus, Lactobacillus brevis, Streptococcus thermophilus, Butyrivibrio fibrisolvens, Rhodococcus jostii, Trichoderma reesei, an Amycolatopsis sp., an Acinetobacter sp., Cellulomonas fimi, Cellulomonas flavigena, Sphingomonas paucimobilis, Streptomyces coelicolor, Irpex flavus and an Acetobacter sp.

A microbial mixture or a microbial inoculant of the invention can then be applied to a collection of leftover plant material from a post-harvest commercial crop or garden residuals. Contacting leftover plant material with a microbial mixture or a microbial inoculant of the invention (for example by mixing it with or applying it to the plant material) transforms the post-harvest materials through degradation by the microbial composition of microbial species. Degradation by the microbial species occurs enzymatically and makes nutrients within the plant material more accessible to the microbes and the environment for recycling purposes. These nutrients are essential for plant growth and include nitrogen, phosphorus, and potassium. The invention provides a method for recycling plant material comprising the steps of applying a microbial inoculant to plant material to form an inoculated plant material, and enclosing (e.g. covering, sealing, placing in a container) the inoculated plant material for at least about two weeks to form a recycled plant material. The method may also include the step of applying the recycled plant material to a live plant, seed, field, greenhouse, or other grow space. The methods of applying microbial inoculant disclosed in this application are direct approaches, introducing precise microbial species that are most effective in recycling wasted Cannabis and other high-lignin-content plant material into bioavailable nutrients to nourish new plants. In addition to creating a microbial community that produces a consistent output, a microbial inoculant and a method of the invention also involves less manual labor and accelerates recycling speed.

A microbial recycling inoculant can be poured, sprayed, or otherwise applied to post-harvest residuals compiled to form a plant material. Compiled plant material can be run through a chipper, or otherwise ground using methods known in the art to reduce size of plant material and increase surface area. In some instances, post-harvest residuals are from a harvest of Cannabis. In other instances, post-harvest residuals are from a harvest of other commercial crops, including but not limited to hops, ornamental flowers (roses, orchids, lavender, lilies, geranium, marigold), saffron, nursery and/or landscaping clippings from trees and bushes, Christmas trees (fir trees), wine grapes, sunflowers, broccoli, rice, tomatoes, sugar cane, corn, wheat, soy, cotton, home garden residuals, deciduous leaves, palm fronds, and tea (C. sinensis var. sinensis and C. s. var. assamica).

A compiled plant material can be placed in a tarp, lined pit, or otherwise enclosed container when the microbial recycling inoculant is applied. The plant material can be left covered for about 2-8 weeks or more, but depending on temperature and weather conditions even up to 4-6 months as well as the timing for application and use, to allow for the development of an environment to facilitate the microbial activity (such as a microaerobic or an anaerobic environment) and degradation of plant material for recycling purposes. This can occur at the site of extraction or processing of desirable plant products, or, having transported the leftover plant materials back to fields, a location proximal to fields outside of an extraction or processing site. This can occur either indoors or outdoors. After the recycling period, recycled plant material can then be applied back to live plant, seed, field, greenhouse, or other grow space as a nutrient source.

Alternatively, stalks and unused plant material can be left in fields following harvest. These leftover plant materials can be mowed, cutdown, or knocked over, and, preferably broken down or frayed in the process to reduce size of plant material and increase surface area. A microbial recycling inoculant can be sprayed or otherwise applied to the leftover plant materials in fields. Leftover plant materials and microbial recycling inoculant can be harrowed into fields or covered in soil for about 2-8 weeks or more, but depending on temperature and weather conditions even up to 4-6 months, to facilitate the microbial activity and degradation of plant materials for recycling purposes.

A microbial mixture or microbial inoculant of the invention can be used for retting. Natural retting is a process by which hemp bast fiber is separated from the woody core through allowing the stalks to rot in the field and be broken down by weather and exposure to natural elements. Retting degrades compounds like pectin and lignin from the long fibers allowing them to be easily separated from one another and from the inner core of the stalk. Retting is commonly used for processing hemp but can also be used to process other bast-type plants, including but not limited to: flax, hemp, hay, straw, and ramie; wild plants, such as stinging nettle; and trees such as lime or linden, willow, oak, wisteria, and mulberry. The fibrous exterior, bast, is used for clothing, other woven or non-woven goods, and more; whereas the wood-like interior, hurd, is used for paper, hempcrete, insulation, and more. Plants are cut, and the stalks are spread on the ground and exposed to water via sprinklers or natural means, such as rain or dew. Typically, natural microbial activity aids in the process. In one aspect of the invention, a microbial mixture or microbial inoculant of the invention can be applied to plant stalks spread on the ground or left in a field to facilitate the retting process. Accordingly, a microbial mixture or microbial inoculant of the invention can be useful for degrading compounds like pectin and lignin. In one embodiment, a microbial mixture or microbial inoculant used for retting applications comprises L. casei, L. plantarum, L. fermentum, L. delbruecki, B. subtilis, S. cerevisiae, R. palustris, P. chrysosporium, I. lacteus, and P. putida.

### EXAMPLES

### Example 1: Lyophilized Microbial Mixture Production—General Protocol

Bacterial and/or fungal strains are inoculated from frozen glycerol stock or solid growth plate into 5-500 mL liquid media appropriate for each strain. Strains are inoculated from 5-500 mL liquid culture into 20-50 L liquid culture. Cultures are cycled through draw/fill growth cycles as desired. Strains are pelleted by centrifugation and combined into a single 2 L media suspension. The combined 2 L suspension is Lyophilized. These steps are repeated for each individual microbial species that is to comprise the inoculant. All Lyophilized species are combined to generate a final lyophilized microbial composition. Final concentrations of inoculant species are equal compared to one another and at least OD₆₀₀=0.7 (0.7×10⁸ CFU/mL). Lyophilized inoculant is packaged as packets for subsequent use.

Nutrient media for each microbe is comprised of the components as described in Table 1.

TABLE 1

| Microbes | Media Name | Media Components |
|---|---|---|
| Bacillus subtilis | Nutrient | Beef extract |
| Rhodopseudomonas palustris | | Peptone |
| Pseudomonas putida | | |
| Rhodococcus jostii | | |
| Sphingomonas paucimobilis | | |
| Cellulomonas fimi | | |
| Cellulomonas flavigena | | |
| Lactobacillus casei | MRS | Meat extract |
| Lactobacillus plantarum | | Glucose |
| Lactobacillus fermentum | | Yeast extract |
| Lactobacillus delbrueckii | | Peptone |
| Lactobacillus acidophilus | | Mg²⁺ and Mn²⁺ sulfates |
| Lactobacillus brevis | | Ammonium citrate |
| | | Sodium acetate |
| Saccharomyces cerevisiae | Yeast Mold | Yeast extract |
| Saccharomyces pastorianus | | Malt extract |
| Trichoderma reesei | | |
| Phanerochaete chrysosporium | | Dextrose |
| Irpex lacteus | | Peptone |
| Acetobacter | Mannitol | Yeast extract |
| | | Peptone |
| | | Mannitol |
| Streptococcus thermophilus | Tryptic Soy | Tryptone |
| Butyrivibrio fibrisolvens | | Soytone |
| Acinetobacter spp. | | NaCl |
| Amycolatopsis spp. | Yeast Malt Extract | Yeast extract |
| Streptomyces coelicolor | | Malt extract |
| | | Peptone |
| | | Dextrose |

### Example 2: Preparation of a Microbial Recycling Inoculant

### 2.1 General Resuspension Protocol:

Lyophilized packet is resuspended in 1 gallon of water. Optionally, this suspension is supplemented with a sugar and/or carbon source. This forms the “microbial inoculant” for recycling purposes.

### Example 3: Application of Recycling Inoculant

### 3.1 Recycling of Collected Post-Harvest Residuals:

Stalks and other leftover plant material are collected in a pile. Stalks are run through a wood-chipper or other grinding mechanism to increase surface area. Chipped plant material is placed onto a tarp, into a container, or into a lined pit. The microbial inoculant is applied to the plant material. The plant material is covered with a tarp or enclosed in container or pit for 2-4 weeks of microbial activity. Recycled plant material can then be applied back to fields or to plants in greenhouse or other grow spaces as a nutrient source.

### 3.2 Recycling of Post-Harvest Residuals Left in Field:

Stalks and unused plant material are left in fields following harvest. Stalks are mowed, cut down, or knocked over in the fields and ideally broken down or frayed in the process to increase surface area. Microbial inoculant is sprayed onto stalks and leftover plant material left in fields. Stalks and leftover plant material are harrowed into fields or covered in soil for at least 2-4 weeks.

### 3.3 Recycling of Post-Extraction Residuals in Piles:

Post-extraction waste is collected and transported back to fields, to a location outside the extraction facility, or to a centralized processing location. Post-extraction plant material is placed onto a tarp, into a container, or into a lined pit. The microbial inoculant is applied to the plant material. The plant material is covered with a tarp or enclosed in container or pit for 2-4 weeks of microbial activity. Recycled plant material can then be applied back to fields or to plants in greenhouse as a nutrient source.

### 3.4 Recycling of Post-Extraction Residuals in Containers:

Post-extraction plant material is collected and placed into containers. The microbial inoculant is applied to plant material in containers. Containers are sealed for 2-4 weeks of microbial activity. Recycled plant material can then be applied back to fields or to plants in greenhouse as a nutrient source.

### Example 4: Ecoenzyme Assays

### 4.1 Microbial Inoculant and Recycled Biomass Production

Four microbial inoculants (FCM1-4) were prepared and tested at several time points and 5 samples per time point. The microbes comprising each inoculant were grown individually in 500 mL liquid culture and combined at an equal ratio based on OD_{600 nm} to create a final liquid culture with an OD_{600 nm}=1 and ˜3 L total volume. This solution was then added directly to 40 g shredded hemp in a 1:1 (v/w) ratio. Samples were left in covered containers at room temperature to produce recycled biomass. The microbe composition of the inoculants was as follows:
FCM1: Lactobacillus casei, Lactobacillus plantarum, Lactobacillus fermentum, Lactobacillus delbrueckii, Bacillus subtilis, Saccharomyces cerevisiae, Rhodopseudomonas palustris, and Phanerochaete chrysosporium.
FCM2: Lactobacillus casei, Lactobacillus plantarum, Lactobacillus fermentum, Lactobacillus delbrueckii, Bacillus subtilis, Saccharomyces cerevisiae, Rhodopseudomonas palustris, and Irpex lacteus.
FCM3: Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, and Irpex lacteus.
FCM4: Lactobacillus casei, Lactobacillus plantarum, Lactobacillus fermentum, Lactobacillus delbrueckii, Bacillus subtilis, Saccharomyces cerevisiae, Rhodopseudomonas palustris, Phanerochaete chrysosporium, Irpex lacteus, and Pseudomonas putida.

### 4.2 Ecoenzyme Assays

Hydrolase, oxidase, amino-peptidase, and esterase activity was quantified as indicators of microbial functional activity and expressed as nmol h⁻¹ g⁻¹ of dry recycled biomass. Hydrolases (BG=β-glucosidase and NAG=β-1,4-N-acetylglucosaminidase) serve as an indicator for hydrolysis of plant and fungal cell walls, respectively. Oxidase (peroxidase and phenol oxidase), L-leucine aminopeptidase (LAP), and phosphatase (PP) activity are indicators for degradation of lignin, proteins, and phosphate, respectively. Sample suspensions were prepared by adding 0.5 g recycled biomass to 100 mL of 50 mM, pH 7.0, sodium bicarbonate buffer and homogenizing for 90 s with a Brinkman Polytron. The microplates were organized to assay three samples per plate, with two columns of eight wells each, for 16 replicates for each sample, along with controls (250 mL buffer alone, 200 mL buffer with 50 mL reference, and 200 mL buffer with 50 mL substrate). The reference standard was a 50-mM solution. Substrates were prepared as 200-mM solutions in nanopure (18.2 megaohm) water. Microplates were covered and incubated at 20° C. for 2 h. After incubation, they were quantified using a microplate fluorimeter (FLx800, Bio-Tek Instruments) with 360-nm excitation and 460-nm emission filters.

Oxidative enzyme substrates consisted of 50 mM L-DOPA for the phenol oxidase assay and 50 mM L-DOPA with 0.3% hydrogen peroxide for the peroxidase assay. The plates were covered and incubated for 1.5 h at 20° C. Absorbance was read on a microplate spectrophotometer with a 520-nm filter. Actual oxidative activity is the sum of phenol oxidase and peroxidase.

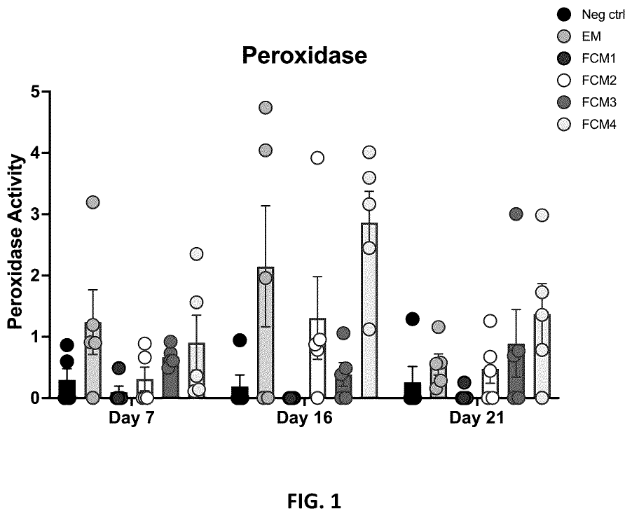

Ecoenzyme assay results are shown in FIGS. 1-6. FCM1-4 treated samples were compared to EM (state-of-the-art industry standard) treated and to untreated control.

FIG. 1 indicates that versions of FCM outperform the state-of-the-art in a peroxidase activity assay. Peroxidase is an enzyme responsible for degrading lignin. Negative control represents shredded hemp grown without any added microbes, though there are some residual microbes present from the environment. FCM4 peroxidase activity outperformed EM at Day 16 and Day 21 and was similar to EM at Day 7.

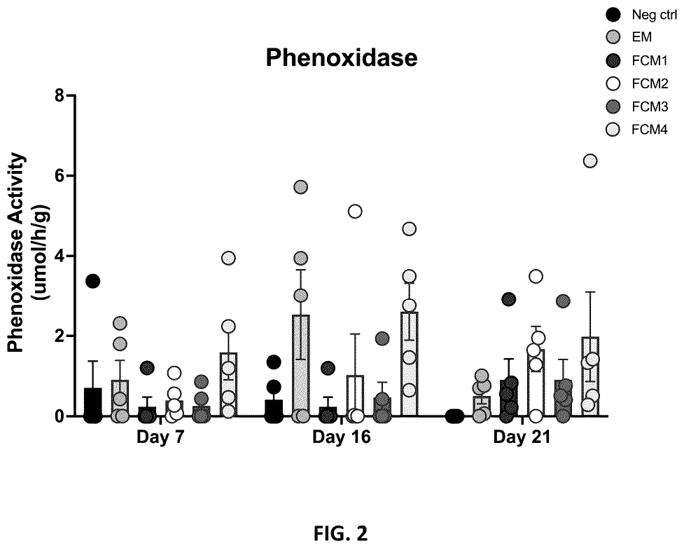

FIG. 2 indicates versions of FCM outperform the state-of-the-art in a phenoxidase activity assay. Phenoxidase is an enzyme responsible for degrading aromatic substances including lignin. Negative control represents shredded hemp grown without any added microbes, though there are some residual microbes present from the environment. FCM4 phenoxidase activity outperformed EM at Day 7 and Day 21 and was similar to EM at Day 16. Note the large bimodal distribution of samples in the Day 7, Day 16, and Day 21 EM conditions, with half of the samples showing no phenoxidase activity.

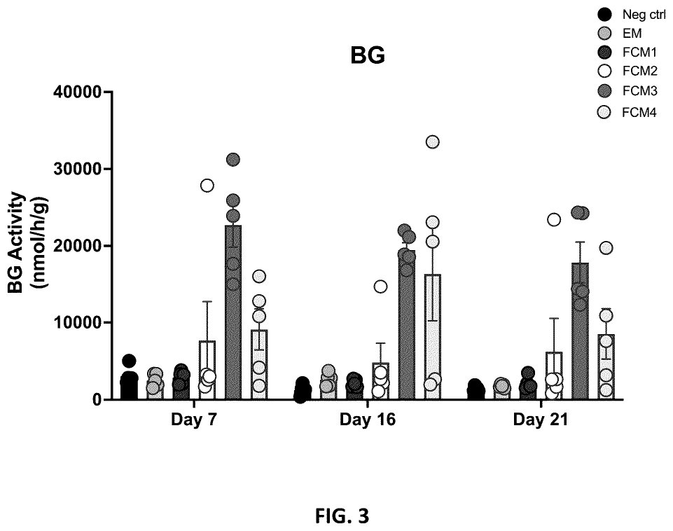

FIG. 3 indicates that versions of FCM outperform the state-of-the-art in a β-glucosidase activity assay. β-glucosidase is an enzyme responsible for releasing glucose from compounds such as cellulose. Negative control represents shredded hemp grown without any added microbes, though there are some residual microbes present from the environment. FCM3 and FCM4 outperformed EM at all days measured.

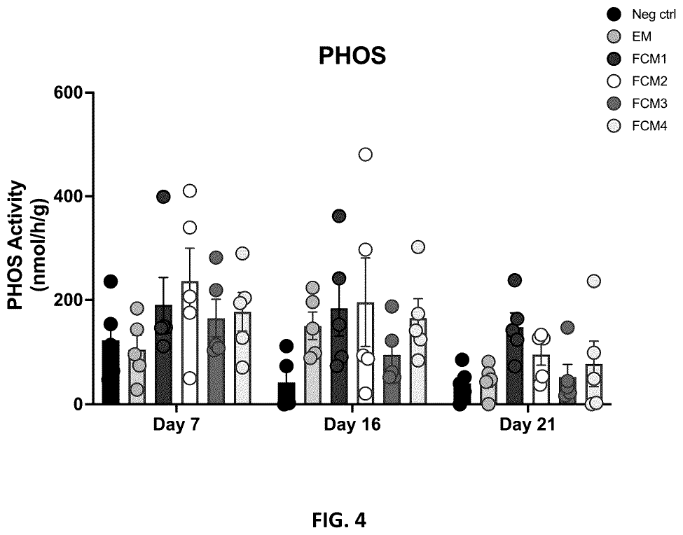

FIG. 4 indicates that versions of FCM outperform the state-of-the-art in a phosphatase activity assay. Phosphatase, an enzyme responsible for releasing phosphate from chemical bonds, was measured using the phosphatase assay. Negative control represents shredded hemp grown without any added microbes, though there are some residual microbes present from the environment. FCM1, FCM2, and FCM4 outperformed EM at all days measured, though activity is low in general.

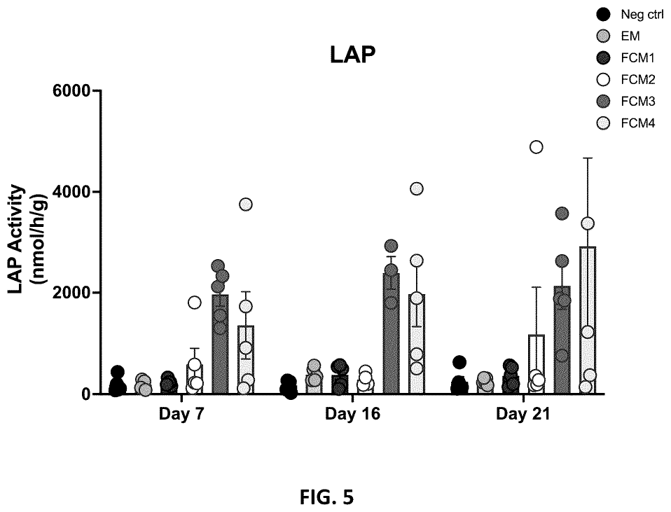

FIG. 5 indicates that versions of FCM outperform the state-of-the-art in a leucyl-aminopeptidase (LAP) activity assay. LAP, is an enzyme responsible for hydrolyzing hydrophobic amino acids. Negative control represents shredded hemp grown without any added microbes, though there are some residual microbes present from the environment. FCM3 and FCM4 outperformed EM at all days measured. EM production of LAP was minimal to low at all days tested.

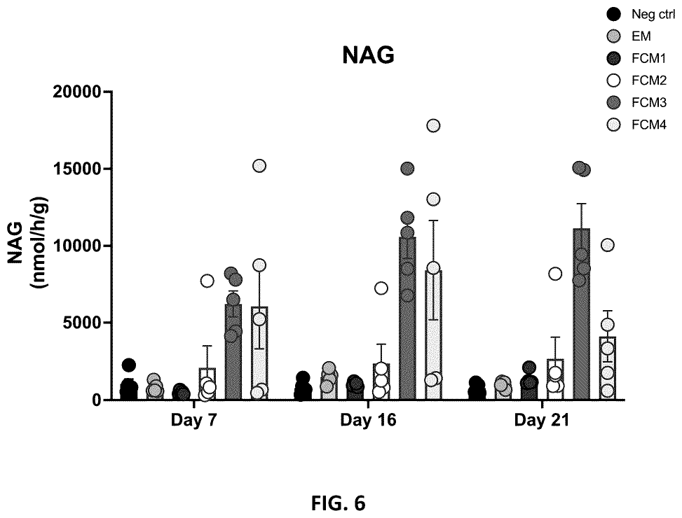

FIG. 6 indicates that versions of FCM outperform the state-of-the-art in a N-acetylglutamate synthase (NAG) activity assay. NAG is an enzyme responsible for hydrolyzing N-containing amino acids. Negative control represents shredded hemp grown without any added microbes, though there are some residual microbes present from the environment. FCM3 and FCM4 outperformed EM at all days measured. EM production of NAG was minimal to low at all days tested.

### Example 5: Pathogen Suppression/Plate Competition Assay

Inoculant and recycled biomass preparation was as described in Example 4. Where indicated, samples underwent a further curing process wherein following the recycling process, the biomass was cured for 1-2 weeks by mixing recycled biomass and coco coir or soil at a 1:1 ratio (v/v) in a 5-gallon bucket. The bucket was closed and left untouched for 1 week for coco coir and 2 weeks for soil, though the seal was not air-tight.

A half gram of recycled biomass was added to 50 mL of sterile water and shaken overnight. The next day, 1.5 g agar was added to 50 mL deionized water and autoclaved for 30 min. It was cooled to 55° C., mixed in with the recycled biomass water extract, swirled gently to mix, and poured into 100 mm×15 mm plastic petri plates. The next day, plugs of Rhizoctonia solanii growing on potato dextrose agar were transferred onto the recycled biomass water extract plates, and pure water agar plates were used as a control. Plates were incubated for 24 h at room temperature. The mycelium radius was then measured to the nearest 1 mm using a microscope. Three of the longest radii were recorded, and the mean was used as a representative measure to compare suppressive potential among different recycled biomass samples. This assay was completed five times in replicate per recycled biomass type. All measurements were standardized against the control of mean mycelium radial growth on water agar.

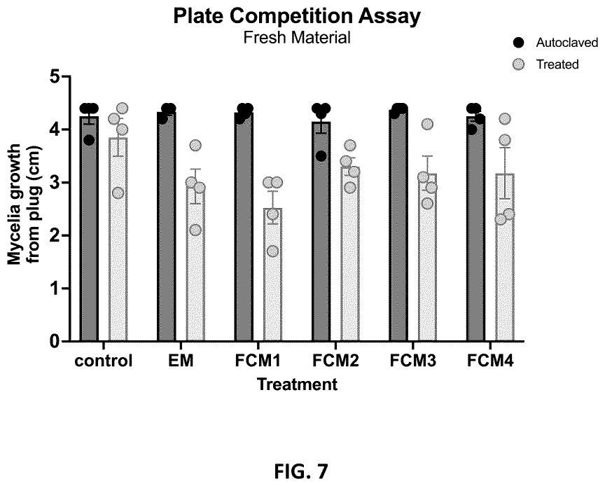

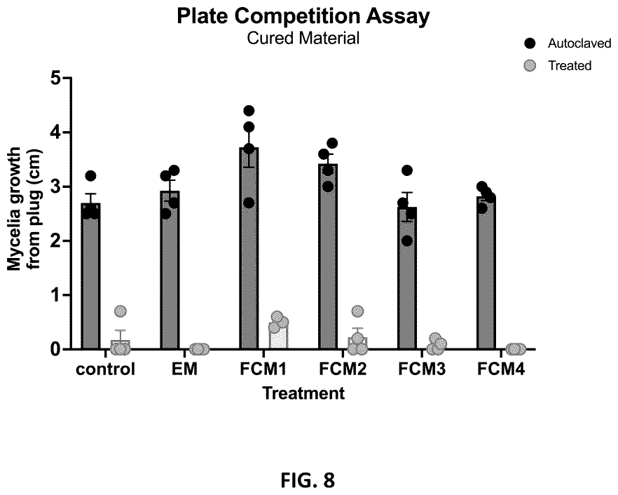

Plate competition assay results are shown in FIGS. 7-8. FCM1-4 treated samples were compared to EM (state-of-the-art industry standard) treated and to untreated control.

FIG. 7 indicates that fresh biomass recycled with different versions of FCM have pathogen suppression activity. Pathogen suppression was measured using a plate competition assay wherein a plug of Rhizoctonia solanii was used as the pathogen. Fresh recycled biomass was added to the plate (“Treated”), and autoclaved recycled biomass was used as a negative control. A lower mycelial growth from plug indicates more pathogen suppression by the treatment. The 4 versions of FCM had similar levels of pathogen suppression compared to the state-of-the-art EM.

FIG. 8 indicates that cured recycled biomass treated with different versions of FCM have pathogen suppression activity. Pathogen suppression was measured using a plate competition assay. A plug of Rhizoctonia solanii was used as the pathogen. Cured recycled biomass was added to the plate (“Treated”), and autoclaved recycled biomass was used as a negative control. A lower mycelial growth from plug indicates more pathogen suppression by the treatment. The 4 versions of FCM had similar levels of pathogen suppression compared to the state-of-the-art EM. These data also indicate the importance of curing the recycled biomass rather than using it fresh.

### Example 6: Germination Bioassay

Inoculant and recycled biomass preparation and curing process was as described in Examples 4 and 5. Recycled biomass-soil mixtures were allowed to equilibrate for 1 week after which 25 radish seeds were planted into each pot using a customized dibble-stick to ensure a distance of 254 mm between each seed. Four replicate pots were ascribed to each treatment sample. Plant bioassays were performed in the greenhouse under natural day lengths and watered daily. Cress seedlings were allowed to grow for 2 weeks until the emergence of one true pair of leaves, after which germination rate was determined for each bioassay.

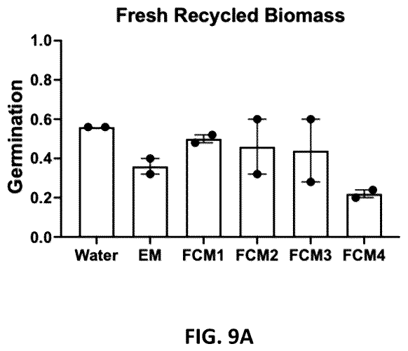

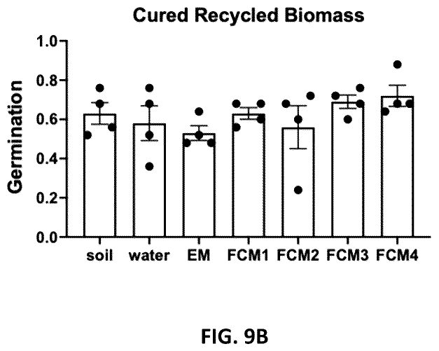

Germination bioassay results are shown in FIGS. 9A and 9B. A negative control of no recycled biomass and EM (state-of-the-art industry standard) treated soil served as references.

FIG. 9A-B indicates that cured recycled biomass treated with different versions of FCM increase seedling germination compared to fresh recycled biomass. Whereas fresh recycled biomass treated with FCM4 does not outperform EM, cured recycled biomass treated with FCM4 has a much greater germination rate, indicating that curing the recycled biomass is a key implementation step. Upon curing, versions of FCM all outperform EM in the germination assay. Each data point represents the germination rate of one pot of 25 seeds.

### Example 7: Cress Growth in Recycled Biomass in Coco Coir

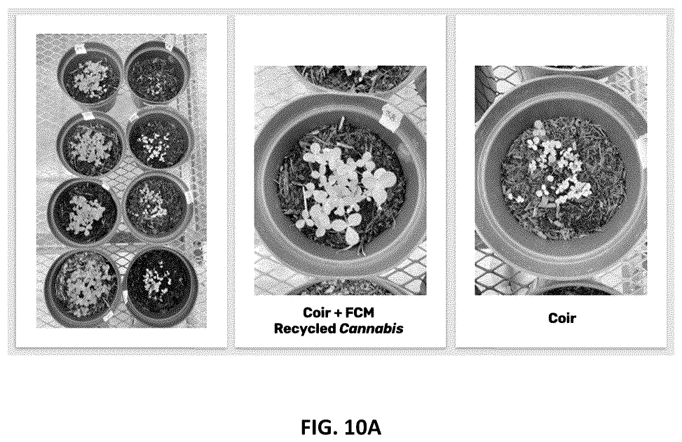

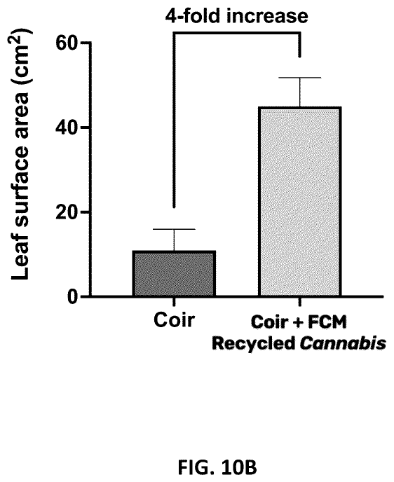

Inoculant and recycled biomass preparation and curing process was as described in Examples 4 and 5. Shredded hemp was recycled for 3 weeks with FCM4 and then cured for 1 week by creating a 1:1 mixture of recycled biomass and coco coir (Coir+FCM Recycled Cannabis). Results, shown in FIG. 10A, demonstrate recycled biomass cured in coco coir results in more vibrant, larger, healthier cress seedlings compared to seedlings in coco coir alone. Cress seeds (n=25/pot) were planted in the mixture or the coco coir and tracked through germination.

Following germination, leaf surface area was quantified using ImageJ software (FIG. 10B; 21, 22). Briefly, analysis in ImageJ begins by adjusting the scale of the image using the object of known size. The image is then converted into 8-bit Color, and the Threshold is adjusted to provide the software with a baseline value to determine what is leaf tissue and what is background. Next, “Area” is selected from the pre-programmed list in ImageJ, each individual leaf is then manually selected, and ImageJ calculates the surface area. These data indicate that cress seedlings planted in recycled biomass+coir were 4-fold larger compared to cress seedlings planted in coir alone.

### Example 8: Nutrient Analysis of Soil Treated with Recycled Hemp

### 8.1 Preparation of FCM4-Recycled Hemp and Soil Treatment

Inoculant and recycled biomass preparation and curing process was as described in Examples 4 and 5. Shredded hemp was recycled for 2 weeks with FCM4 and then cured for 2 weeks by creating a 1:1 mixture of recycled biomass and soil. Control-recycled hemp was generated by mixing water with shredded hemp for 2 weeks, and then cured for 2 weeks by mixing 1:1 with soil (“Base soil”).

### 8.2 Nutrient Analysis

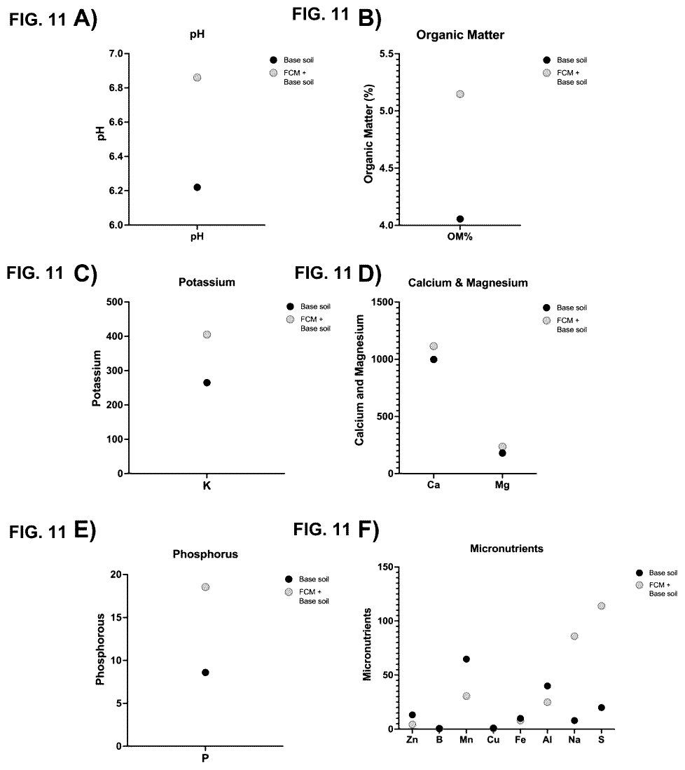

Nutrient analysis for each sample was performed to determine pH, organic matter percentage, K, Ca & Mg, P, and micronutrients including Zn, B, Mn, Cu, Fe, Al, Na, and S as described (23). Results, shown in FIGS. 11A-11F, indicate that recycling inoculant-treated samples had increased pH, increased organic matter, and increased K, Ca & Mg, P, Na, and S compared to control-treated soil.

### Example 9: Nutrient Analysis of Soil Treated with Bran Recycled Hemp

### 9.1 Preparation of FCM4 Bran Recycled Hemp

FCM-bran was generated by mixing one-part FCM4, one part bran, one part molasses, and 100 parts water for 2 weeks, after which shredded hemp was mixed in. This mixture of FCM4 bran and shredded hemp was recycled for 2 weeks. Following the 2-week recycling period, the mixture was cured for 2 weeks with soil (“FCM Bran+Base soil”). The above methods were repeated with the current state-of-the-art inoculant EM to create EM Bran+Base soil. Control-recycled hemp was generated by mixing water with shredded hemp for 2 weeks, and then cured for 2 weeks by mixing 1:1 with soil (“Base soil”).

### 9.2 Nutrient Analysis

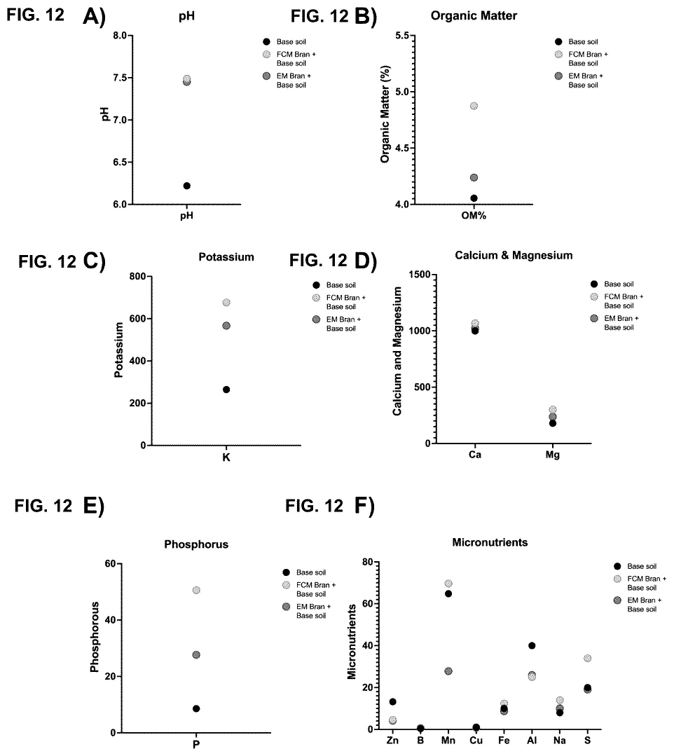

Nutrient analysis for each sample was performed to determine pH, organic matter percentage, K, Ca, Mg, P, and micronutrients including Zn, B, Mn, Cu, Fe, Al, Na, and S as described in Example 8. Results shown in FIGS. 12A-12F show that FCM Bran-treated samples had increased pH, increased organic matter, and increased K, Ca & Mg, P, Mn, Na, and S compared to control-treated soil. FCM Bran-treated samples had increased pH, increased organic matter, and increased K, Ca & Mg, P, Mn, Fe, Na, and S compared to EM Bran+Base soil.

### Example 10: Lignin Quantification of In-Field Recycled Rye Straw

Stalks and unused rye straw were recycled as described in Example 3.2. Mown rye straw was left untouched or raked into windrows (raked), sprayed with recycling inoculant, and left in the field to be recycled for 3 weeks.

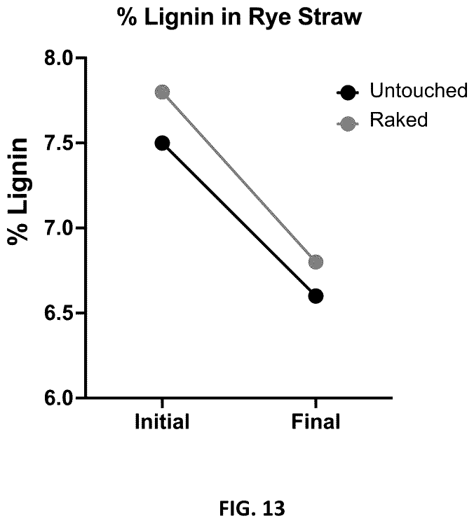

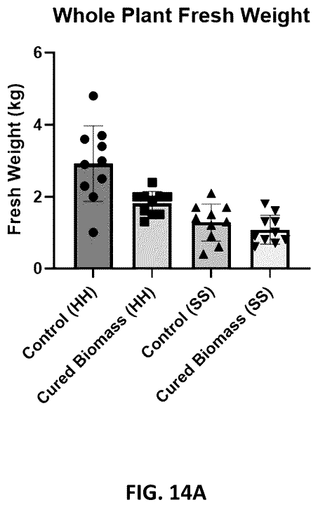

A sample of rye straw from each condition was collected at the beginning of recycling (initial) and 3 weeks later (final), when recycling was complete. Samples were analyzed for lignin concentration using the Neutral Detergent Fiber Analysis after amylase treatment method (24). In both conditions, lignin concentration decreased over 3 weeks as shown in FIG. 13.

### Example 11: In-Field Hemp Plant Trial

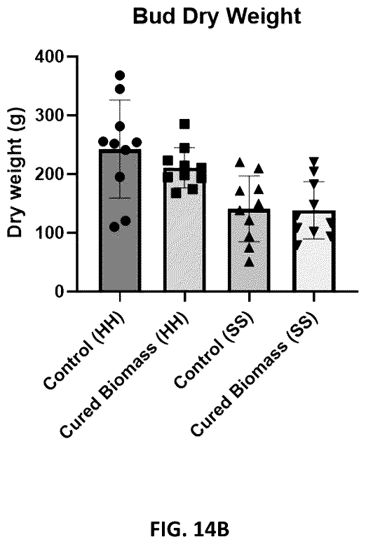

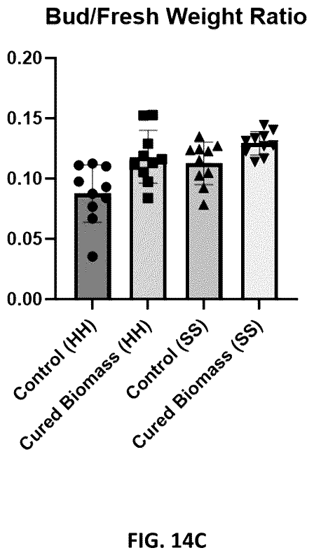

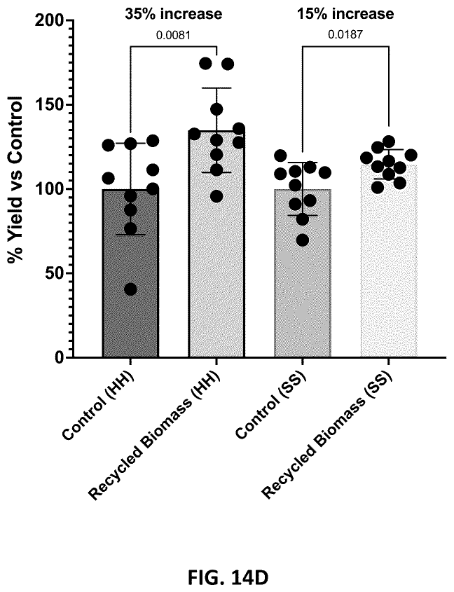

Stalks and unused hemp biomass were recycled as described in Example 3.1, with minor modifications. Briefly, unused hemp biomass was treated with recycling inoculant for 2 weeks, covered with a tarp, and then run over with a tractor to shred the recycled hemp into smaller pieces. Recycled biomass was dispersed throughout a growing field and tilled into the ground to cure, after which 2 hemp varieties, Sour Hawaiian Haze (HH) and Sour Special Sauce (SS) were transplanted into the field and allowed to grow over the grow season. Hemp plants were also transplanted into untreated fields. After reaching maturity, hemp plants were harvested, and whole plant fresh weight was measured. Plants were dried, buds were harvested, and bud dry weight was measured. With these data, bud/fresh weight ratio and % Yield versus control were calculated, indicating that treatment with the cured biomass increased bud/fresh weight ratio and % Yield versus control for both the Sour Hawaiian Haze and Sour Special Sauce varieties, as shown in FIGS. 14A-14D.

### Example 12: Recycling of Non-Hemp Lignin-Rich Inputs

### 12.1 Preparation of Recycled Biomass

Unused plant biomass from a variety of non-hemp lignin-rich inputs was used to create an assortment of recycled biomasses. In particular, palm leaves, rice husks, coconut husks, peanut shells, grape vines, corn leaves and stalks, poplar chips, and flower trimmings were used to make recycled biomass. These lignin-rich inputs were treated with FCM4 as described in Examples 4 and 5. The recycled biomasses were then cured with soil for 2 weeks as described in Example 8.

### 12.2 Nutrient Analysis

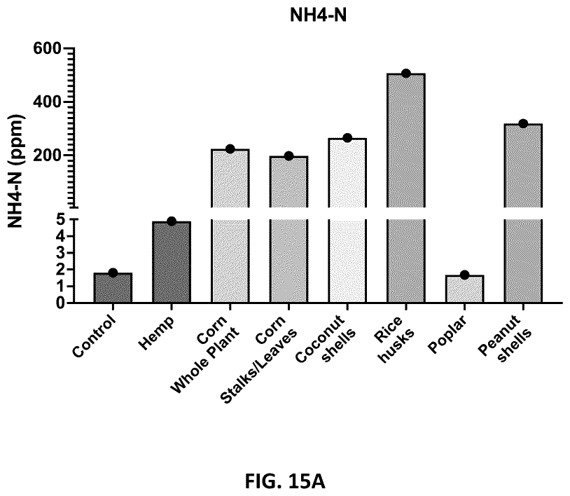

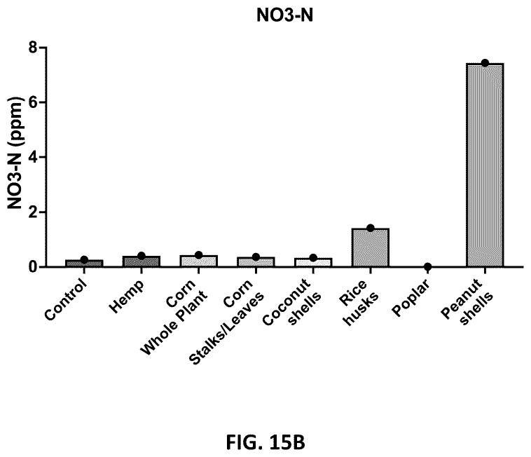

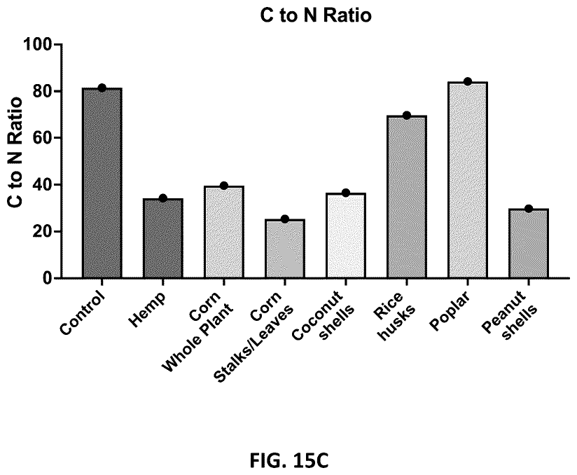

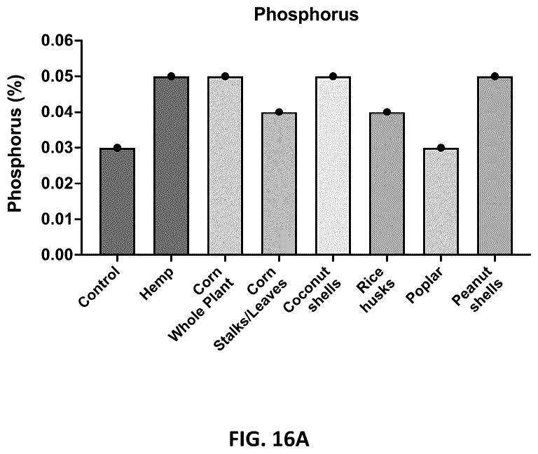

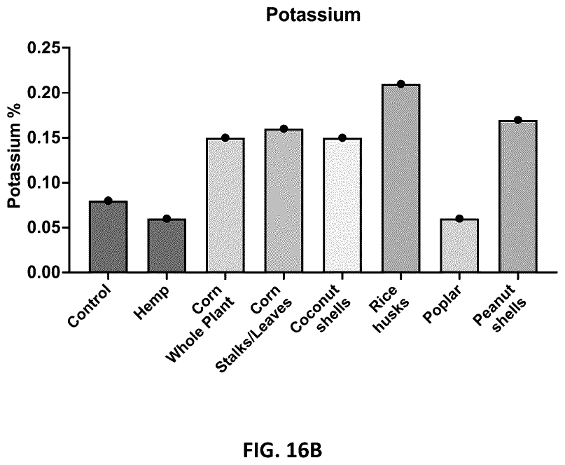

Nutrient analysis was performed as described in Example 8. The data, shown in FIGS. 15A-15C, indicate that NH₄—N and NO₃—N are released from the assorted lignin-rich material during recycling compared to the control samples, causing a decrease in the C to N ratio. A C to N ratio of 30:1 is considered to be ideal for nutrient supplementation (25). Additionally, phosphorus and potassium content were analyzed and are shown in FIGS. 16A-16B. These data indicate that phosphorus and potassium are released from the various lignin-rich material during recycling compared to the control samples.

### Example 13: Retting

Plants are cut, and the stalks are spread on the ground and exposed to water via sprinklers or natural means, such as rain or dew. Microbial inoculants are prepared as described in Examples 1 and 2. The inoculant is applied at a rate of 2 gallons of inoculant at OD=1, diluted into water to spray 1 acre, but can be diluted more or less. The retting process can take about 3 to about 45 days depending on the weather, and occurs between about 40-100° F.

### REFERENCES

1. IBIS. 2019. Fertilizer Manufacturing Industry in the US.
2. FAOSTAT. 2019. Food and Agricultural Organization of the United Nations. http://www.fao.org/faostat/en/#data. Accessed November 2019.
3. EPA. 2017. Inventory of U.S. Greenhouse Gas Emissions and Sinks. Agency USEP, EPA. https://www.epa.gov/sites/production/files/2019-04/documents/us-ghg-inventory-2019-main-text.pdf.
4. BrightfieldGroup. 2019. US Hemp Cultivation Landscape.
5. Mark T, Shepherd J. 2019. Industrial Hemp Budgets. University of Kentucky College of Agriculture.
6. Unkovich M J, Baldock J A, Forbes M. 2006. A review of biological yield and harvest index in Australian field crops, p 30. In Sparks D L (ed), Advances in Agronomy, vol 105.
7. Zhu L, O'Dwyer J P, Chang V S, Granda C B, Holtzapple M T. 2008. Structural features affecting biomass enzymatic digestibility. Bioresour Technol 99:3817-3828.
8. de Gonzalo G, Colpa D I, Habib M H, Fraaije M W. 2016. Bacterial enzymes involved in lignin degradation. J Biotechnol 236:110-119.
9. Kim Y K, Lee S C, Cho Y Y, Oh H J, Ko Y H. 2012. Isolation of Cellulolytic Bacillus subtilis Strains from Agricultural Environments. ISRN Microbiol 2012:650563.
10. Aro N, Pakula T, Penttila M. 2005. Transcriptional regulation of plant cell wall degradation by filamentous fungi. FEMS Microbiol Rev 29:719-739.
11. Kim J H, Block D E, Mills D A. 2010. Simultaneous consumption of pentose and hexose sugars: an optimal microbial phenotype for efficient fermentation of lignocellulosic biomass. Appl Microbiol Biotechnol 88:1077-1085.
12. Stevulova N, Cigasova J, Estokova A, Terpakova E, Geffert A, Kacik F, Singovszka E, Holub M. 2014. Properties Characterization of Chemically Modified Hemp Hurds. Materials (Basel) 7:8131-8150.
13. Heard J, Watson K, Kostiuk J. 2019. Nutrient Uptake and Partitioning by Industrial Hemp. http://www.hemptrade.ca/eguide/production/nutrient-use. Accessed 2019.
14. Blake A W, Marcus S E, Copeland J E, Blackburn R S, Knox J P. 2008. In situ analysis of cell wall polymers associated with phloem fibre cells in stems of hemp, Cannabis sativa L. Planta 228:1-13.
15. Neher D A, Weicht T R, Bates S T, Leff J W, Fierer N. 2013. Changes in bacterial and fungal communities across compost recipes, preparation methods, and composting times. PLoS One 8:e79512.
16. McNear Jr. DH. 2019 2013. The Rhizosphere—Roots, Soil, and Everything in Between. 4(3).
17. Coker C. Feb. 1, 2019 2019. Composting and Microbial Inoculants. 60(2).
18. Neher D A, Fang L, Weicht T R. 2017. Ecoenzymes as Indicators of Compost to Suppress Rhizoctonia solani. Compost Science & Utilization 24:251-261.
19. Salvachda D, Karp E M, Nimlos C T, Vardon D R, Beckham G T. 2015. Towards lignin consolidated bioprocessing: simultaneous lignin depolymerization and product generation by bacteria. Green Chemistry 17:16.
20. Bugg T D, Ahmad M, Hardiman E M, Singh R. 2011. The emerging role for bacteria in lignin degradation and bio-product formation. Curr Opin Biotechnol 22:394-400.
21. onlinelibrary.wiley.com/doi/10.1111/j.1365-3040.2012.02498.x
22. rookieecologist.wordpress.com/2016/11/21/how-to-measure-leaf-area-in-imagej/
23. Recommended Soil Testing Procedures for the Northeast United States. Northeastern Regional Publication No. 493, 3rd edition. Revised Jul. 1, 2011.
24. cfh.uni-hohenheim.de/en/amylase
25. compost.css.cornell.edu/chemistry.html

The claimed invention is:

1. A method of retting plant material, comprising the step of contacting the plant material to be retted with a mixture of microbial species to form an inoculated plant material,
wherein the mixture of microbial species comprises, consists essentially of, or consists of:
at least one first microbial species selected from the group consisting of Lactobacillus acidophilus, Saccharomyces pastorianus, Lactobacillus brevis, Streptococcus thermophilus, Butyrivibrio fibrisolvens, Pseudomonas putida, Rhodococcus jostii, Trichoderma reesei, Phanerochaete chrysosporium, Irpex lacteus, Bacillus subtilis, Amycolatopsis spp., Acinetobacter spp., Cellulomonas fimi, Cellulomonas flavigena, Sphingomonas paucimobilis, Streptomyces coelicolor, and Irpex flavus; and
at least one second microbial species selected from the group consisting of Lactobacillus casei, Lactobacillus plantarum, Lactobacillus fermentum, Lactobacillus delbrueckii, Bacillus subtilis, Saccharomyces cerevisiae, Rhodopseudomonas palustris, and Acetobacter spp.

2. The method of retting according to claim 1, wherein:
a) the at least one first microbial species is Phanerochaete chrysosporium, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris;
b) the at least one first microbial species is Irpex lacteus, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris;
c) the at least one first microbial species are Phanerochaete chrysosporium and Irpex lacteus, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris; or
d) the at least one first microbial species are Phanerochaete chrysosporium, Irpex lacteus, and Pseudomonas putida, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris.

3. The method of retting according to claim 2, wherein the at least one first microbial species are Phanerochaete chrysosporium, Irpex lacteus, and Pseudomonas putida, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris.

4. Use of a microbial inoculant for retting, wherein the microbial inoculant comprises, consists essentially of, or consists of:
at least one first microbial species selected from the group consisting of Lactobacillus acidophilus, Saccharomyces pastorianus, Lactobacillus brevis, Streptococcus thermophilus, Butyrivibrio fibrisolvens, Pseudomonas putida, Rhodococcus jostii, Trichoderma reesei, Phanerochaete chrysosporium, Irpex lacteus, Bacillus subtilis, Amycolatopsis spp., Acinetobacter spp., Cellulomonas fimi, Cellulomonas flavigena, Sphingomonas paucimobilis, Streptomyces coelicolor, and Irpex flavus;
at least one second microbial species selected from the group consisting of Lactobacillus casei, Lactobacillus plantarum, Lactobacillus fermentum, Lactobacillus delbrueckii, Bacillus subtilis, Saccharomyces cerevisiae, Rhodopseudomonas palustris, and Acetobacter spp.;
water; and
an optional carbon source.

5. The use of a microbial inoculant according to claim 3, wherein:
a) the at least one first microbial species is Phanerochaete chrysosporium, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris;
b) the at least one first microbial species is Irpex lacteus, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris;
c) the at least one first microbial species are Phanerochaete chrysosporium and Irpex lacteus, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris; or
d) the at least one first microbial species are Phanerochaete chrysosporium, Irpex lacteus, and Pseudomonas putida, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris.

6. The use of a microbial inoculant according to claim 4, wherein the at least one first microbial species are Phanerochaete chrysosporium, Irpex lacteus, and Pseudomonas putida, and the at least one second microbial species are Lactobacillus casei, Lactobacillus plantarum, Lactobacillus fermentum, Lactobacillus delbrueckii, Bacillus subtilis, Saccharomyces cerevisiae, and Rhodopseudomonas palustris.
